(19) United States
(12) Patent Application Publication
(10) Pub. No.: US 2018/0280537 A1
(43) Pub. Date: Oct. 4, 2018

(54) MULTIFUNCTIONAL POLYANIONIC CYCLODEXTRIN DENDRIMERS

(71) Applicant: UTI LIMITED PARTNERSHIP, Calgary (CA)

(72) Inventors: Chang-Chun LING, Calgary (CA); John KLASSEN, Edmonton (CA); Emma-Dune LERICHE, Bordeaux (FR); Ping ZHANG, Calgary (CA); Aixia WANG, Calgary (CA)

(21) Appl. No.: 15/766,785

(22) PCT Filed: Oct. 7, 2016
(86) PCT No.: PCT/CA2016/051177

§ 371 Date: Apr. 6, 2018

Related U.S. Application Data
(60) Provisional application No. 62/238,616, filed on Oct. 7, 2015.

(51) Int. Cl. A61K 47/69; A61K 47/56

(52) U.S. Cl. CPC A61K 47/6951; A61K 47/56

(57) ABSTRACT

The present application provides a multifunctional cyclodextrin dendrimer of the formula comprising at least one anionic residue and at least one non-ionic residue bound to the cyclodextrin ring structure, and methods of their manufacture. The multifunctional cyclodextrin dendrimer as described herein can be used for drug delivery.

### FIELD

The present application pertains to the field of cyclodextrins. More particularly, the present application relates to one-pot synthesis of libraries of multifunctional polyanionic cyclodextrin dendrimers for targeted and non-targeted drug delivery.

### BACKGROUND

Cyclodextrins (CDs) are a class of non-toxic, water-soluble D-glucose based macrocycles with a hydrophobic cavity. CDs typically vary by the number of glucose units. Common members include α-CD (6 glucose units), β-CD (7 glucose units) and γ-CD (8 glucose units), with increasing cavity size. The varying cavity sizes offer increased utility in a wide variety of applications, particularly in drug delivery models. For example, CDs can be used to form “inclusion complexes” in which a drug is included and carried within the cavity. This can be used as a pharmaceutical excipient to improve drug water solubility, chemical stability, and removal of certain drug side effects (such as undesirable taste). CDs have also drawn interest in the cosmetic and food additives industries, in the design of artificial enzymes, gene delivery vehicles, sensors and novel supramolecular assemblies.

CDs can be native or chemically modified on either or both of their primary and/or secondary faces. Typically, an inclusion complex often has lower water solubility than native CDs. Chemical modifications of CDs can change their physico-chemical properties. For example, adding a p-toluenesulfonyl (tosyl) group on the primary face of the β-CD renders the molecule near insoluble in water at room temperature, while adding methyl groups at OH-6 and OH-2 positions significantly increases water solubility. The toxicity of the molecule can also be changed. Therefore, modification of the CD molecule may present certain advantages.

Adding charged functionalities to a cyclodextrins via a linker has been known to effectively improve their water solubility. A typical example is to add sulfobutyl ether groups to beta-cyclodextrin, creating a library of cyclodextrin derivatives (SBEBCD) for use in drug formulations.

However, the charged groups repel each other because of the presence of the same charges; this could push the linkers to be away from reach other, reducing the inclusion efficiency.

In addition, current commercial SBEBCD derivatives are prepared using partially deprotonated beta-cyclodextrin as a nucleophile in basic conditions, to react with 1,4-butane sultone as the electrophile. This leads to the formation of a series of SBEBCD derivatives that bear the sulfobutyl ether residues randomly distributed at both the primary face and secondary face of beta-cyclodextrin.

It is therefore more desirable to include both charged groups and non-charged groups to a cyclodextrin to obtain hybrid molecules containing both charged and non-charged groups; such molecules are expected to have improved water-solubility and binding affinity with the guest molecule, because along with linker of charged arms, the non-charged groups are not repelled by charged groups and, thus, they can also efficiently interact with included guest molecules.

In addition, targeting group such as a bioactive carbohydrate, biotin, folic acid, etc. could be chemically attached to non-ionic group, creating cyclodextrin derivatives with targeting ability to biological receptors such as lectin, streptavidin, folate receptors etc.

Moreover, improved chemistries are desired to generate cyclodextrin derivatives with better-defined geometry, such as placing all substituents at only once face of cyclodextrins. This creates a new generation of chemically modified cyclodextrin hosts that can maximize the cooperative effects among introduced groups when interacting with included guest (drug) molecules.

This background information is provided for the purpose of making known information believed by the applicant to be of possible relevance to the present invention. No admission is necessarily intended, nor should be construed, that any of the preceding information constitutes prior art against the present invention.

### SUMMARY

An object of the present invention is to provide a multifunctional modified cyclodextrin dendrimer to provide enhanced drug delivery functionality.

In accordance with an aspect of the present invention, there is provided a multifunctional cyclodextrin dendrimer of the structure:

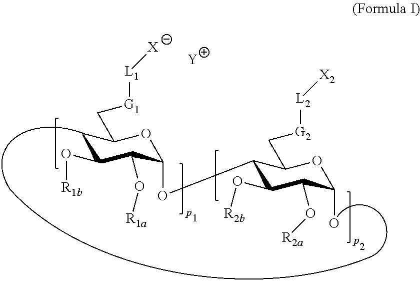

wherein
X⁽⁻⁾ is one or more negatively charged moieties,
Y⁽⁺⁾ is one or more counter cations,
X₂ is one or more neutral moieties,
L₁ and L₂ are each one or more linkers,
G₁ and G₂ are each a bond or are one or more bridging groups,
R₁ₐ, R_{1b}, R₂ₐ and R_{2b} are one or more substituents and can be the same or different, and
each of p1 and p2 is at least 1, and p1+p2=6, 7, or 8;
where if p1>1, each of G₁, L₁, X₍₋₎, Y⁽⁺⁾, can be the same or different from other G₁, L₁, X⁽⁻⁾, Y⁽⁺⁾, respectively; and
where if p2>1, each G₂, L₂, X₂, can be the same or different from other G₂, L₂, X₂, respectively.

In certain embodiments, Y⁽⁺⁾ is Na⁺, or any suitable counter cation; X⁽⁻⁾ is —CO₂— or —SO₃— or PO₃²⁻; G₁ and/or G₂ is —S—; L₁ and/or L₂ is —(CH₂)k-, where k is 1 to 11, optionally 1 to 6;
or L₁ and/or L₂ is

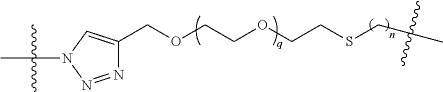

where q is 0 to 20 and n is 1-5, optionally 1-11;
or L₁ and/or L₂ is

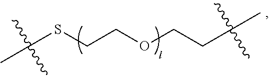

where 1 is 0-20; and R1a, R1b, R2a and R2b are the same or different and are H, optionally substituted C1-C18 alkyl, or optionally substituted C1-C18 acyl.

The linker may be derived from a PEG chain (tetraethylene glycol) but it may also be any non-PEG chain such as an alkyl group of C2-12 length (ethyl to dodecyl) or the combination of PEG and alkyl groups, wherein the alkyl group may or may not be modified.

In certain embodiments, X₂ is a targeting functional group, such as biotin, folic acid, or the like. X₂ can also be any non-targeting polar or nonpolar groups such as —H, —OH, —NR₂, —CO₂NR₂, —CN or the like, where R is H or an alkyl group, typically a C1-C4 alkyl; X₂ can also be a simple carbohydrate such as N-acetyl-lactosamine, D-glucose, D-mannose, N-acetyl-D-glucosamine, L-fucose, N-acetyl-D-glucosamine, N-acetylneuraminic acid or any natural or synthetic oligosaccharide such as lactose and the like.

In certain targeting embodiments, the multifunctional cyclodextrin dendrimer is:

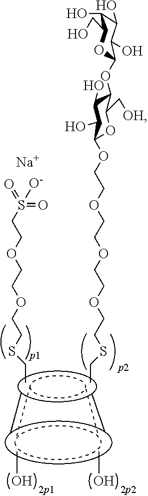

where p1+p2 may be either 6 (α-CD), 7 (β-CD) or 8 (γ-CD). Thus, p1+p2 may be: a) 6, where p1 and p2 are 1 to 5, or more typically 2 to 4; b) 7, where p1 and p2 are 1 to 6, or more typically 2 to 5; or c) 8, where p1 and p2 are 1 to 7, or more typically 2 to 6.

In this embodiment, the linker between the lactose is derived from a PEG chain (tetraethylene glycol) but it can be any non-PEG chain such as an alkyl group of C2-12 length (ethyl to dodecyl) or the combination of PEG and alkyl groups.

In other embodiments, the multifunctional cyclodextrin dendrimer is:

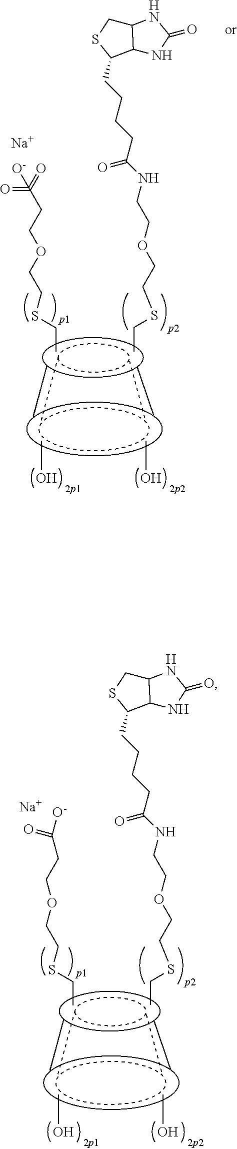

where p1+p2 may be either 6 (α-CD), 7 (β-CD) or 8 (γ-CD). Thus, p1+p2 may be: a) 6, where p1 and p2 are 1 to 5, or more typically 2 to 4; b) 7, where p1 and p2 are 1 to 6, or more typically 2 to 5; or c) 8, where p1 and p2 are 1 to 7, or more typically 2 to 6.

In certain non-targeting embodiments, the multifunctional cyclodextrin dendrimer is:

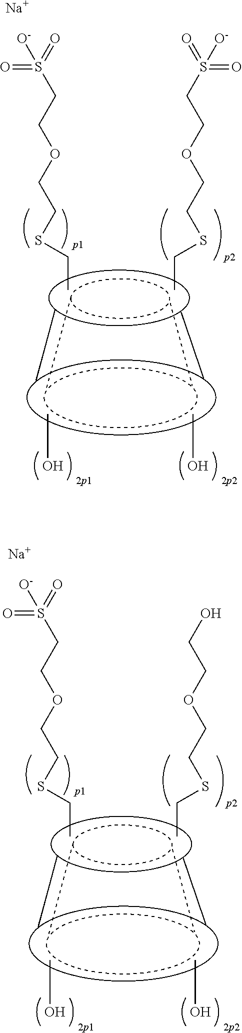

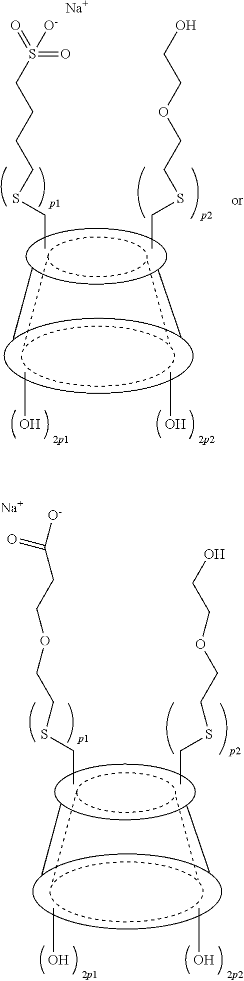

where p1+p2 may be either 6 (α-CD), 7 (β-CD) or 8 (γ-CD). Thus, p1+p2 may be: a) 6, where p1 and p2 are 1 to 5, or more typically 2 to 4; b) 7, where p1 and p2 are 1 to 6, or more typically 2 to 5; or c) 8, where p1 and p2 are 1 to 7, or more typically 2 to 6.

The multifunctional compounds of the present application may have the same or different functional groups therein to facilitate drug delivery.

The present application also provides a method of synthesizing a multifunctional cyclodextrin dendrimer as described herein, comprising: a) providing a per-6-substituted cyclodextrin bearing leaving groups, such as halogen; b) reacting the cyclodextrin derivative in a) with a first residue and a second residue; and c) obtaining the compound, wherein the first and/or second residues comprises one or more linker groups, one or more bridging groups and one or more functional groups as described herein.

The present application also provides a method of drug delivery to a subject comprising administering to said subject a multifunctional cyclodextrin dendrimer as described herein together with the drug. Additionally there is provided a pharmaceutical composition comprising the multifunctional cyclodextrin dendrimer as described herein together with a further compound, such as a drug.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 shows an example of a “native” cyclodextrin compound.

FIG. 2 shows examples of targeting and non-targeting polyanionic multifunctional CD dendrimers for drug delivery as described herein.

FIG. 3 shows an example of a targeting bifunctional library of γ-CD containing sulfonates and lactose residues (compounds 4-8).

FIG. 4 shows an exemplary one-pot synthesis of a bifunctional library of γ-CD (m=8) containing sulfonates and lactose residues and a thioether linkage (compounds 4-8).

FIG. 5 shows an ¹H NMR spectrum of a bifunctional library of γ-CD (m=8) containing compounds 4 to 8.

FIG. 6 shows an electrospray high resolution mass spectrometry (ESI HRMS) spectrum of a bifunctional library of γ-CD (m=8) containing compounds 4 to 8.

FIGS. 7 and 8 provide ESI HRMS characterization of a bifunctional library of γ-CD (m=8) containing compounds 4 to 8.

FIG. 9 shows an example of a selection of non-targeting bifunctional library containing compounds 12-18 that bear the anionic sulfobutyl groups and non-targeting diethylene glycol groups.

FIG. 10 shows an exemplary one pot synthesis of a bifunctional library of γ-CD derivatives (m=8) containing sulfobutyl groups and non-targeting diethylene glycol groups (compounds 12-18).

FIG. 11 shows ¹H NMR spectrum of a bifunctional library of γ-CD (m=8) containing compounds 12-18.

FIG. 12 shows ESI HRMS spectrum of a bifunctional library of γ-CD (m=8) containing compounds 12-18.

FIGS. 13 and 14 show ESI HRMS characterization of a bifunctional library of γ-CD (m=8) containing compounds 12-18.

FIG. 15 shows an example of a selection of non-targeting bifunctional library containing compounds 19-25 that bear both the anionic sulfodiethylene glycol (SulfoDiPEG) and non-targeting diethylene glycol groups.

FIG. 16 shows an exemplary one pot synthesis of the bifunctional library of γ-CD (m=8) containing compounds 19-25 containing both the anionic sulfodiethylene glycol (SulfoDiPEG) and non-targeting diethylene glycol groups.

FIG. 17 shows ESI HRMS spectrum of the non-targeting bifunctional library of γ-CD (m=8) containing compounds 19-25.

FIGS. 18 and 19 show ESI HRMS characterization of the non-targeting bifunctional library of γ-CD (m=8) containing compounds 19-22 (FIG. 18) and 23-25 (FIG. 19).

FIG. 20 shows an example of a selection of non-targeting bifunctional library containing compounds 27-33 that bear both the anionic sulfodiethylene glycol (SulfoDiPEG) and non-targeting hydroxypropyl groups.

FIG. 21 shows an exemplary one pot synthesis of the non-targeting bifunctional library of γ-CD (m=8) containing compounds 27-33.

FIG. 22 shows ¹H NMR spectrum of a bifunctional library of γ-CD (m=8) containing compounds 27-33.

FIG. 23 shows ESI HRMS spectrum of the bifunctional library of γ-CD (m=8) containing compounds 27-33.

FIGS. 24 and 25 show ESI HRMS characterization of the bifunctional library of γ-CD (m=8) containing compounds 27-30 (FIG. 24) and 31-33 (FIG. 25).

FIG. 26 shows an example of a selection of non-targeting bifunctional library containing compounds 35-41 that bear both the anionic sulfodiethylene glycol (SulfoDiPEG) and the anionic sulfobutyl groups. Two different linkers (PEG and butyl) are introduced to the same cyclodextrin scaffold using the one-pot synthesis methodology.

FIG. 27 shows an exemplary one pot synthesis of the non-targeting bifunctional library of γ-CD (m=8) containing compounds 35-41.

FIG. 28 shows an ¹H NMR spectrum of a non-targeting bifunctional library of γ-CD (m=8) containing compounds 35-41.

FIG. 29 shows an ESI HRMS spectrum of the bifunctional library of γ-CD (m=8) containing compounds 35-41.

FIGS. 30 and 31 show ESI HRMS characterization of the bifunctional library of γ-CD (m=8) containing compounds 39-41 (FIG. 30) and 35-38 (FIG. 31).

FIG. 32 shows an example of a selection of targeting bifunctional library containing compounds 42-47 that bear both the anionic sulfodiethylene glycol (SulfoDiPEG) and targeting biotin residues.

FIG. 33 shows an exemplary one pot synthesis of the targeting bifunctional library of γ-CD (m=8) containing compounds 42-47.

FIG. 34 shows ESI HRMS spectrum of the targeting bifunctional library of γ-CD (m=8) containing compounds 42-47.

FIGS. 35 and 36 show ESI HRMS characterization of the bifunctional library of γ-CD (m=8) containing compounds 42-45 (FIG. 35) and 46-47 (FIG. 36).

FIG. 37 shows an example of a selection of non-targeting bifunctional library containing compounds 49-55 that bear both the anionic carboxydiethylene glycol (CarboxyDiPEG) and non-targeting diethylene glycol residues.

FIG. 38 shows an exemplary one pot synthesis of the non-targeting bifunctional library of γ-CD (m=8) containing compounds 49-55.

FIG. 39 shows ESI HRMS spectrum of the non-targeting bifunctional library of γ-CD (m=8) containing compounds 49-55.

FIG. 40 show ESI HRMS characterization of the bifunctional library of γ-CD (m=8) containing compounds 49-55.

FIG. 41 shows an example of a selection of targeting bifunctional library containing compounds 57-62 that bear both the anionic carboxydiethylene glycol (CarboxyDiPEG) and targeting biotin residues.

FIG. 42 shows an exemplary one pot synthesis of the targeting bifunctional library of γ-CD (m=8) containing compounds 57-62.

FIG. 43 shows ESI HRMS spectrum of the bifunctional library of γ-CD (m=8) containing compounds 57-62.

FIGS. 44 and 45 show ESI HRMS characterization of the bifunctional library of γ-CD (m=8) containing compounds 57-60 (FIG. 44) and 61-62 (FIG. 45).

FIG. 46 shows the results of measured dissociation constant (K_{d}) by ESI mass spectrometry of selected bifunctional libraries of γ-CD (m=8) with rocuronium bromide. The selected libraries are those containing compounds 4-8, 12-18, 19-25 and 27-33, respectively.

FIG. 47 shows the results of measured dissociation constant (K_{d}) by ESI mass spectrometry of selected bifunctional libraries of γ-CD (m=8) with doxorubicin hydrochloride. The selected libraries are those containing compounds 57-62.

### DETAILED DESCRIPTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

As used herein, the term “aliphatic” refers to a linear, branched or cyclic, saturated or unsaturated non-aromatic hydrocarbon. Examples of aliphatic hydrocarbons include alkyl groups.

As used herein, the term “alkyl” refers to a linear, branched or cyclic, saturated or unsaturated hydrocarbon group which can be unsubstituted or is optionally substituted with one or more substituent. Examples of saturated straight or branched chain alkyl groups include, but are not limited to, methyl, ethyl, 1-propyl, 2-propyl, 1-butyl, 2-butyl, 2-methyl-1-propyl, 2 methyl 2-propyl, 1 pentyl, 2-pentyl, 3-pentyl, 2-methyl-1-butyl, 3-methyl-1-butyl, 2 methyl-3-butyl, 2,2 dimethyl 1-propyl, 1-hexyl, 2-hexyl, 3-hexyl, 2-methyl-1-pentyl, 3 methyl-1-pentyl, 4 methyl-1-pentyl, 2-methyl-2-pentyl, 3-methyl-2-pentyl, 4 methyl 2 pentyl, 2,2 dimethyl 1 butyl, 3,3-dimethyl-1-butyl and 2-ethyl-1-butyl, 1-heptyl and 1-octyl. As used herein the term “alkyl” encompasses cyclic alkyls, or cycloalkyl groups. The term “cycloalkyl” as used herein refers to a non-aromatic, saturated monocyclic, bicyclic or tricyclic hydrocarbon ring system containing at least 3 carbon atoms. Examples of C3-C12 cycloalkyl groups include, but are not limited to, cyclopropyl, cyclobutyl, cyclopentyl, cyclohexyl, cycloheptyl, cyclooctyl, norbornyl, adamantyl, bicyclo[2.2.2]oct-2-enyl, and bicyclo[2.2.2]octyl. Chemical functional groups, such as ether, thioether, sulfoxide, or amine, amide, ammonium, ester, phenyl, 1,2,3-triazole etc can be incorporated alkyl group to help extend the length of the chain.

As used herein, the term “substituted” refers to the structure having one or more substituents. A substituent is an atom or group of bonded atoms that can be considered to have replaced one or more hydrogen atoms attached to a parent molecular entity. Examples of substituents include aliphatic groups, halogen, arylcarbonyloxy, alkoxycarbonyloxy, aryloxycarbonyloxy, alkoxycarbonyl, aminocarbonyl, alkylthiocarbonyl, alkoxyl, phosphate ester, phosphonato, phosphinato, cyano, tertiary amino, tertiary acylamino, tertiary amide, imino, alkylthio, arylthio, sulfonato, sulfamoyl, tertiary sulfonamido, nitrile, trifluoromethyl, heterocyclyl, aromatic, and heteroaromatic moieties, ether, ester, boron-containing moieties, tertiary phosphines, and silicon-containing moieties.

As used herein, the term “hydrophilic” refers to the physical property of a molecule or chemical entity or substituent within a molecule that tends to be miscible with and/or dissolved by water, or selectively interacts with water molecules. Hydrophilic groups can include polar groups. By contrast, as used herein, the term “hydrophobic” refers to the physical property of a molecule or chemical entity or substituent within a molecule that tends to be immiscible with and/or insoluble in water, or selectively repels water molecules.

As used herein, the term “amphiphilic” refers to the physical property of a molecule or chemical entity that possesses both hydrophilic and hydrophobic properties.

As used herein, the term “anionic” refers to a negatively charged molecule or part thereof which imparts the negative charge.

As used herein, an “excipient” refers to an inactive substance that serves as the vehicle or medium for a drug or other active substance in a pharmaceutical composition.

To gain a better understanding of the invention described herein, the following examples are set forth. It should be understood that these examples are for illustrative purposes only. Therefore, they should not limit the scope of this invention in any way.

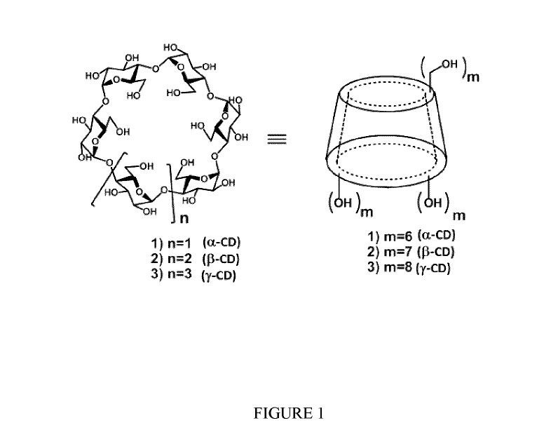

In certain embodiments, the present application provides modified cyclodextrins (CDs), based on native CDs such as those shown in FIG. 1.

The modified CDs as described herein form part of a multifunctional CD library. The CD library comprises modified CDs having at least one anionic residue bound to a monomer of the CD ring structure, and a second residue. As used herein, “residue” is intended to describe a complex comprising: either X⁽⁻⁾, which represents one or more negatively charged moieties, or X₂, which represents one or more neutral moieties, together with L₁ and/or L₂ (which each represent one or more linkers), and G₁ and/or G₂ (which each represents a bond or are one or more bridging groups). Thus, a residue can be anionic (i.e. comprises X(−)-L₁-G₁) or non-ionic (i.e., comprises X₂-L₂-G₂). In any given multifunctional CD compound, there can be any combination of anionic or non-ionic residues, provided there is at least one each of the anionic and non-ionic residues.

A library of multifunctional CDs can contain any number of compounds having p=1 to 7 anionic residues, and 8-p non-ionic residues (for an γ-CD having 8 monomers); p=1 to 6 anionic residues and 7-p non-ionic residues (for a β-CD having 7 monomers); and/or p=1 to 5 anionic residues and 6-p non-ionic residues (for a α-CD having 6 monomers). Thus, there can be any combination of CD compounds of varying size, having any combination of anionic and non-ionic residues. This contributes to the “multifunctional” aspect of the modified CDs in the present application, as there can be any combination of functional features on the varying CDs in the library.

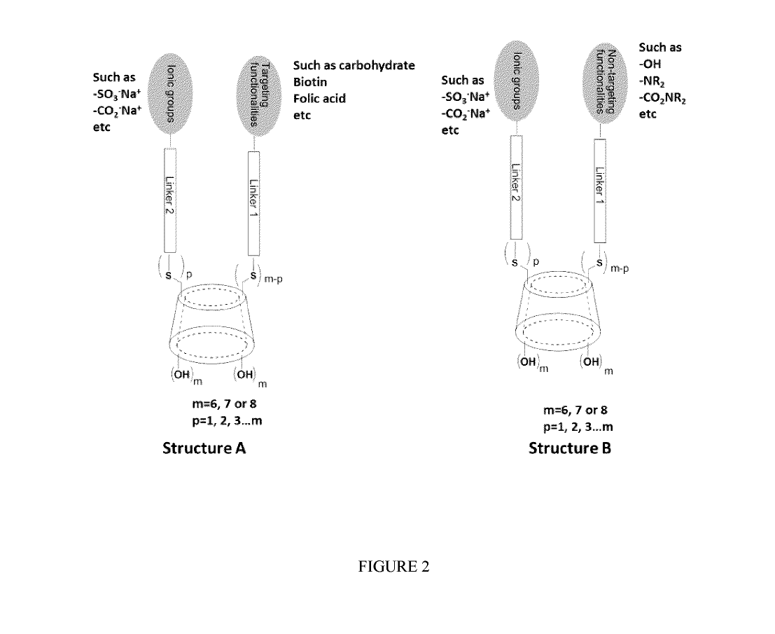

FIG. 2 shows examples polyanionic multifunctional CD dendrimers in the context of the present application. Structure A shows a modified CD having an anionic residue (left) and a “targeting functionality” residue (right). The targeting functionality can include, but is not limited to, biotin, folic acid, lactose, or the like. Structure B shows a modified CD having an anionic residue (left) and a “non-targeting functionality” residue (right). The non-targeting functionality can include a non-ionic functional group including, but not limited to, —H, —OH, —NR₂, —CO₂NR₂, —CN or the like, where R is H or an alkyl group typically containing 1-2 carbons.

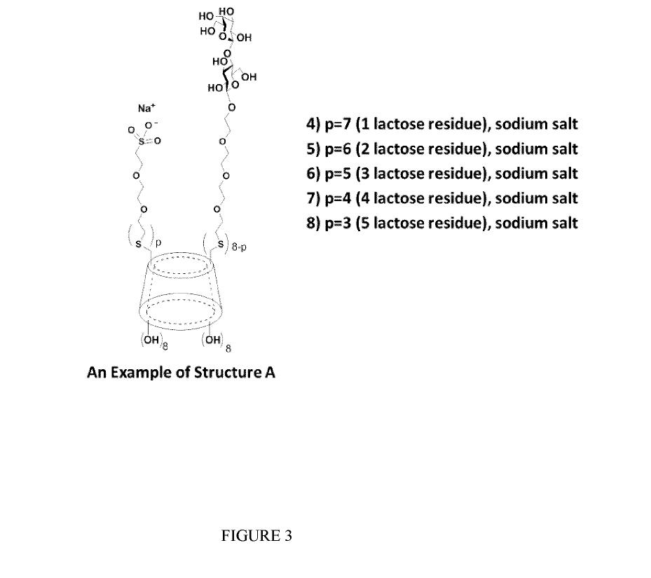

FIG. 3 shows an example of a bifunctional library of CDs β-CD (m=8) containing sulfonates and lactose residues.

### EXAMPLE 1

### One Pot Synthesis of Libraries of Bifunctional Polyanionic Cyclodextrin Dendrimers for Drug Delivery

In certain embodiments, libraries of bifunctional polyanionic cyclodextrin dendrimers can be produced. The libraries can contain a mixture of anionic and non-ionic residues. In this example, the compounds contain groups containing either an anionic residue or a non-ionic residue (“targeting functionalities”). “Multifunctional” as used herein can include bifunctional CDs (i.e., having 2 functions), but can also include those having more than 2 functionalities.

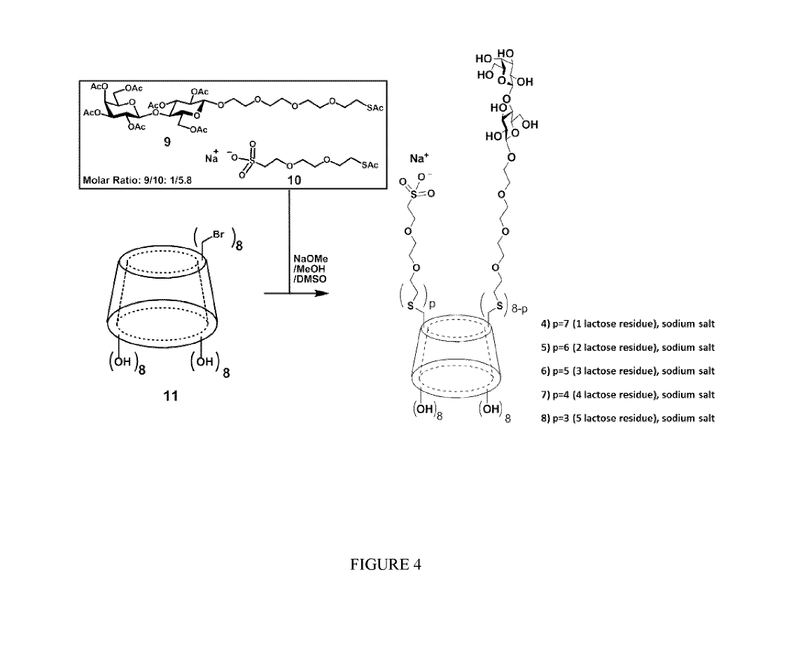

FIG. 4 shows an exemplary one-pot synthesis of a bifunctional library of γ-CD (m=8) containing sulfonates and lactose residues. Typically, a library containing two or more types of reagents bearing the same conjugation functional group are pre-mixed in different ratios; the desired anionic functionality and desired targeting functionality are preinstalled in the reagents. The reagent mixture is then subjected to conjugation with a CD substrate to afford the desired multifunctional library. In the present example, compounds 4, 5, 6, 7 and 8 contain 1, 2, 3, 4 and 5 lactose residues, respectively are obtained, and the generated CD hosts contain a complement of either anionic and/or non-ionic residues, for a total of 8 residues (for a γ-CD having 8 dextrin monomers). Thus, for a compound containing p=1 anionic residue, it would have a corresponding 8-p (i.e., 7) non-ionic (e.g., lactose) residues.

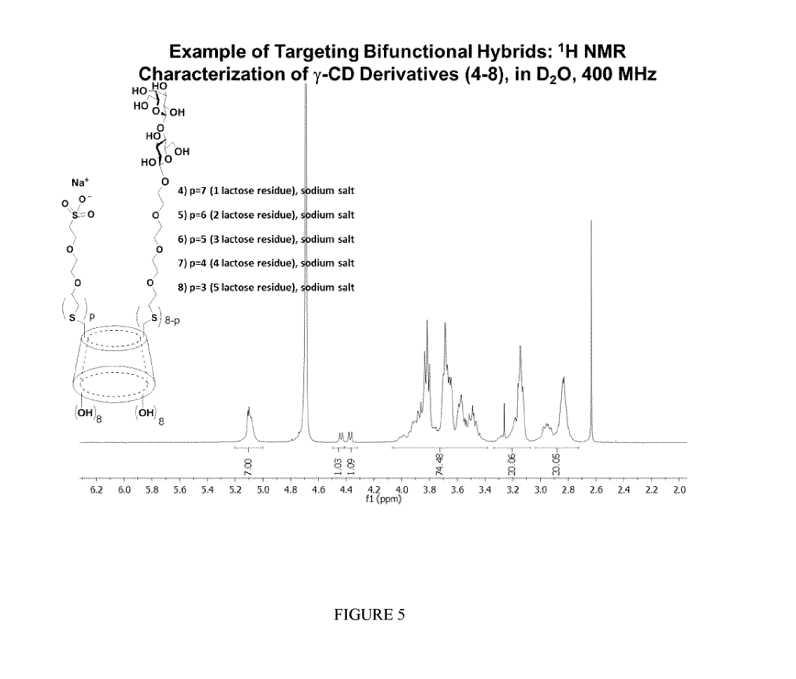

FIG. 5 shows an NMR spectrum of a bifunctional library of γ-CD (m=8) containing compounds 4 to 8.

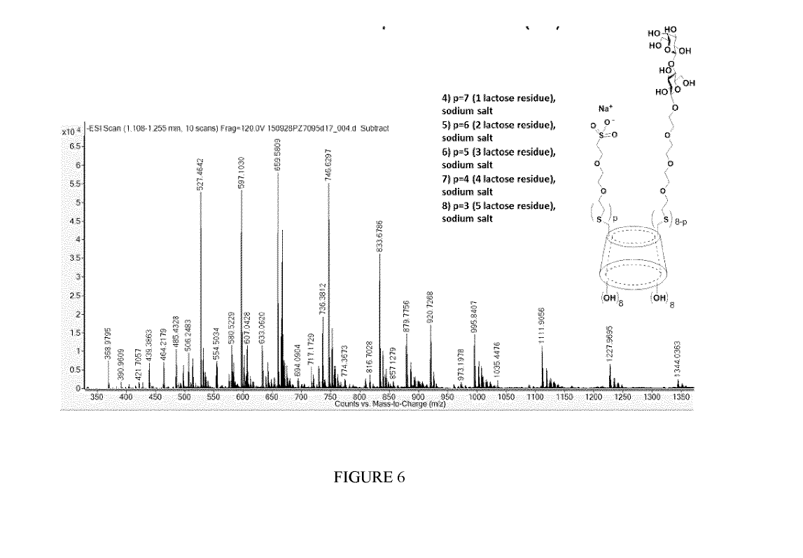

FIG. 6 shows an ESI HRMS spectrum of a bifunctional library of γ-CD (m=8) containing compounds 4 to 8.

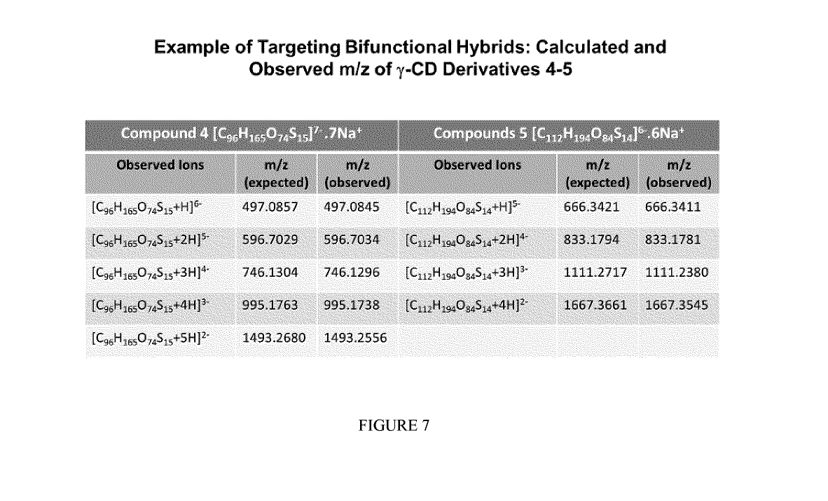

FIG. 7 provides ESI HRMS characterization of a bifunctional library of γ-CD (m=8) containing compounds 4 to 5.

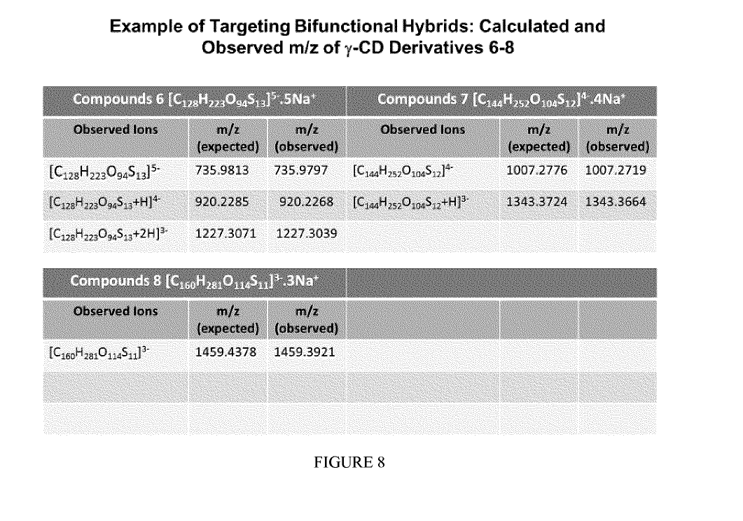

FIG. 8 shows ESI HRMS characterization of a bifunctional library of γ-CD (m=8) containing compounds 6 to 8.

The success and general applicability of the current method is also demonstrated by the synthesis of other targeting bifunctional libraries containing biotin functionality and either sulfonates or carboxylates as the anionic residues.

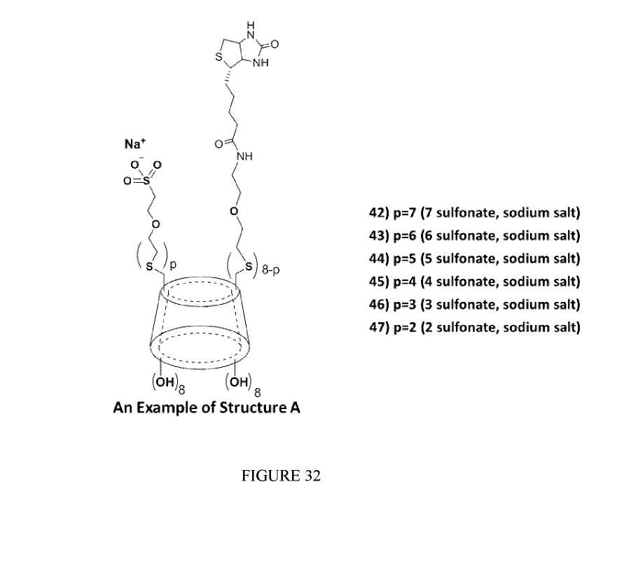

FIG. 32 shows an example of a selection of targeting bifunctional library containing compounds 42-47 that bear both the anionic sulfodiethylene glycol (SulfoDiPEG) and targeting biotin residues.

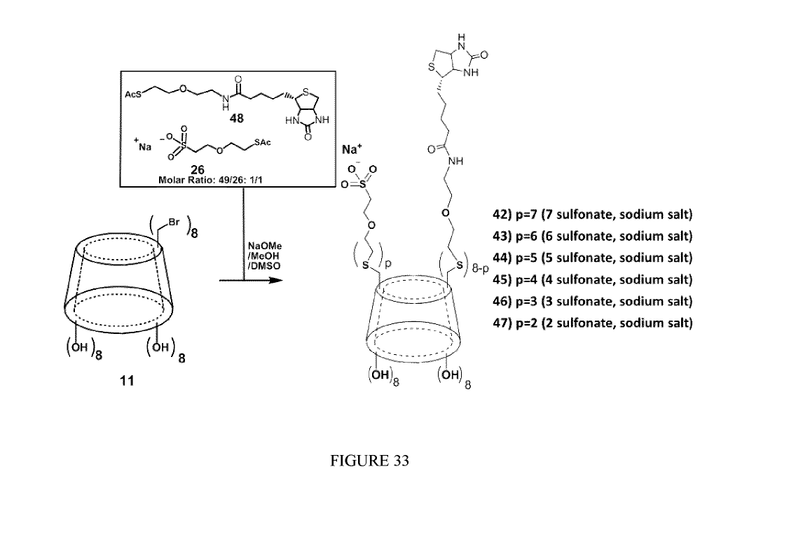

FIG. 33 shows an exemplary one-pot synthesis of the bifunctional library of γ-CD (m=8) containing compounds 42-47.

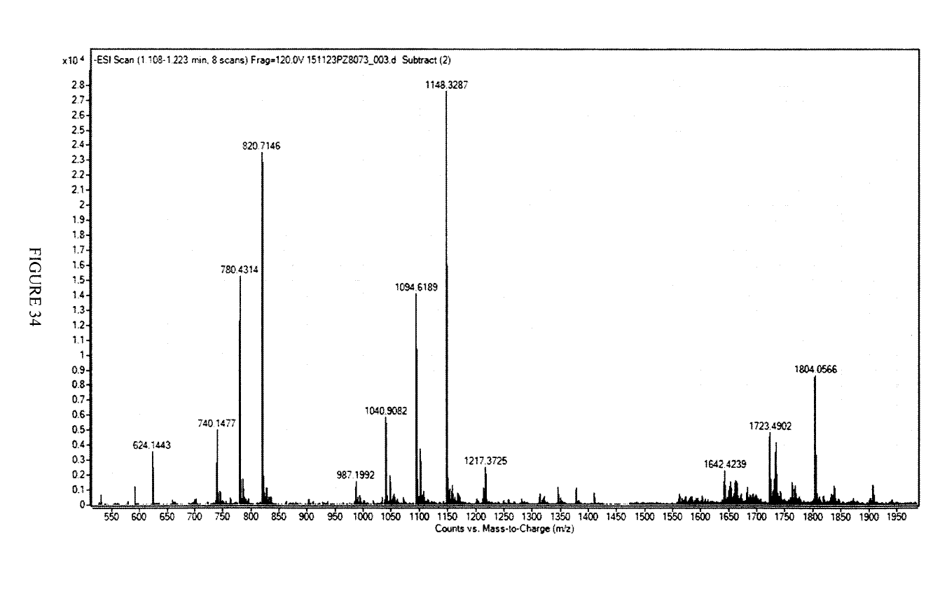

FIG. 34 shows ESI HRMS spectrum of the bifunctional library of γ-CD (m=8) containing compounds 42-47.

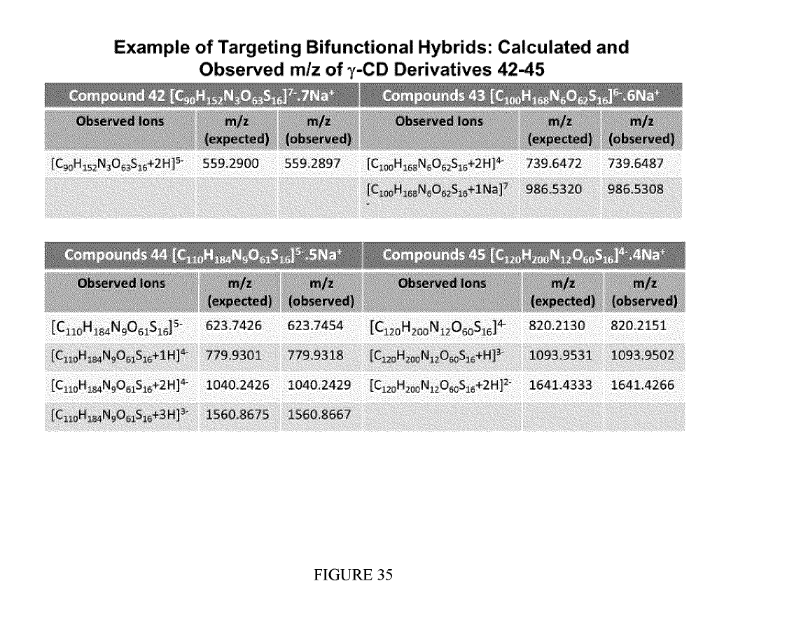

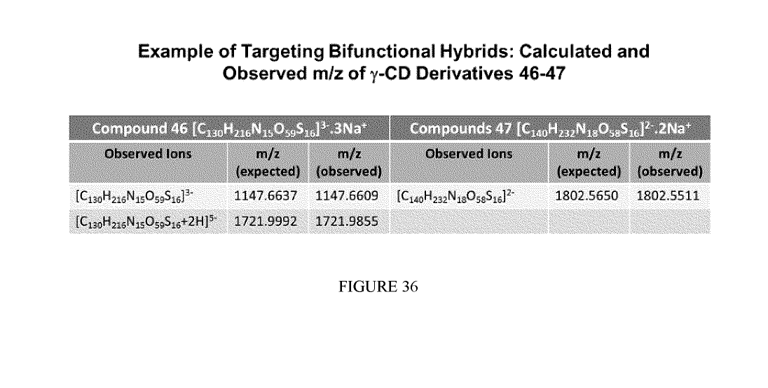

FIGS. 35 and 36 show ESI HRMS characterization of the bifunctional library of γ-CD (m=8) containing compounds 42-47.

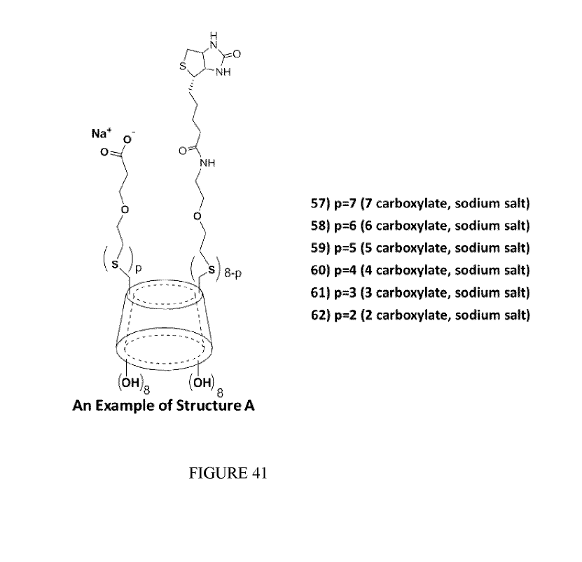

FIG. 41 shows an example of a selection of targeting bifunctional library containing compounds 57-62 that bear both the anionic carboxydiethylene glycol (CarboxyDiPEG) and targeting biotin residues.

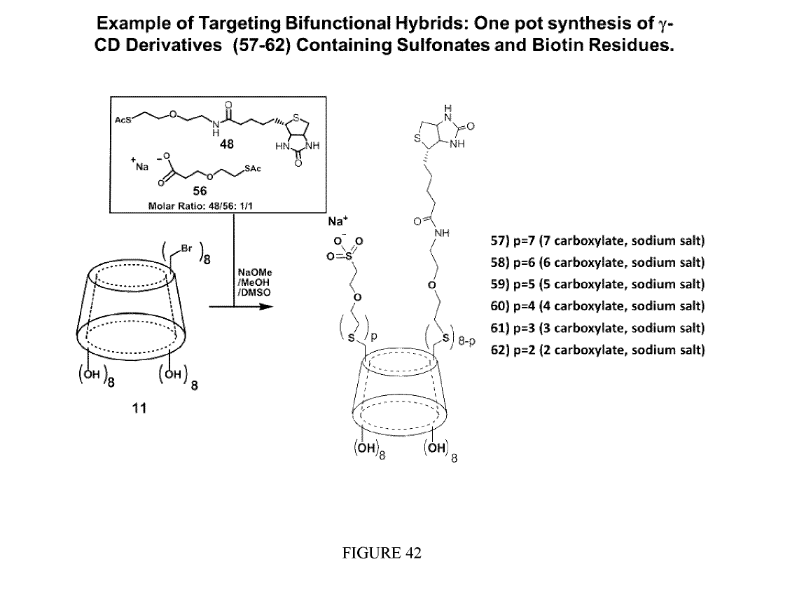

FIG. 42 shows an exemplary synthesis of the bifunctional library of γ-CD (m=8) containing compounds 57-62.

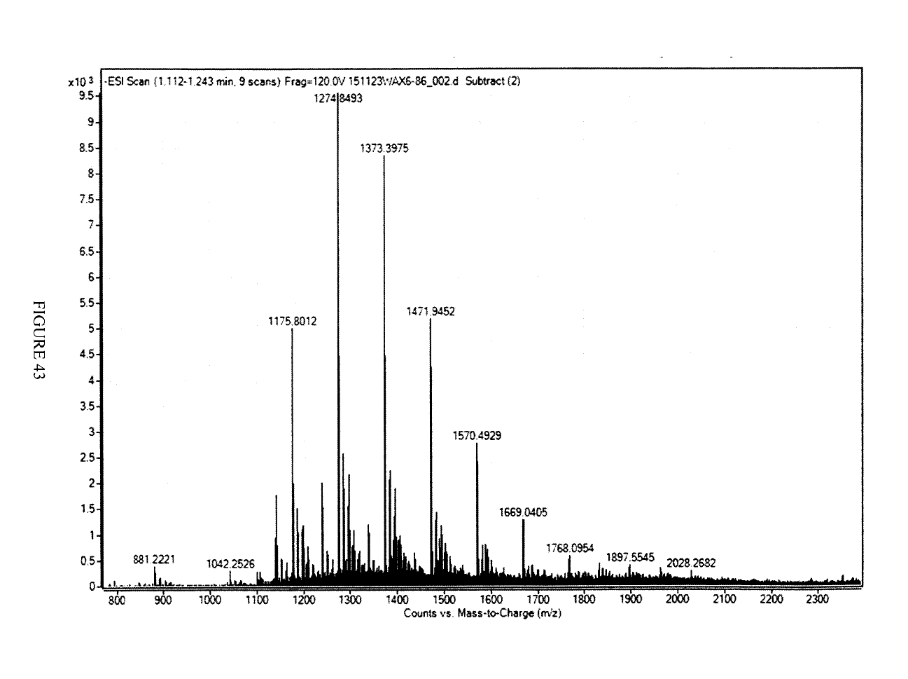

FIG. 43 shows ESI HRMS spectrum of the bifunctional library of γ-CD (m=8) containing compounds 57-62.

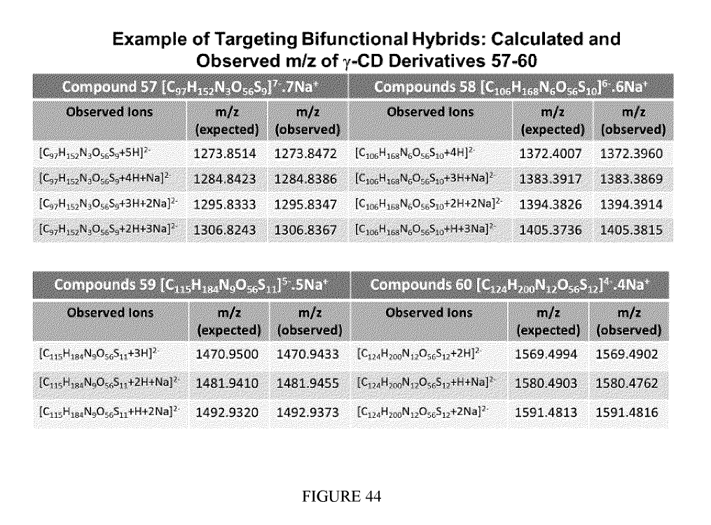

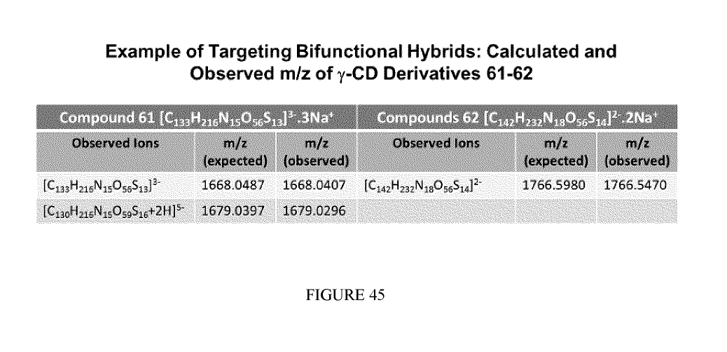

FIGS. 44 and 45 show ESI HRMS characterization of the bifunctional library of γ-CD (m=8) containing compounds 57-62.

### EXAMPLE 2

### Bifunctional Cyclodextrin Libraries Containing Anionic and Non-Targeting Functional Groups

In this example, the CD compounds contain anionic residues and non-targeting residues.

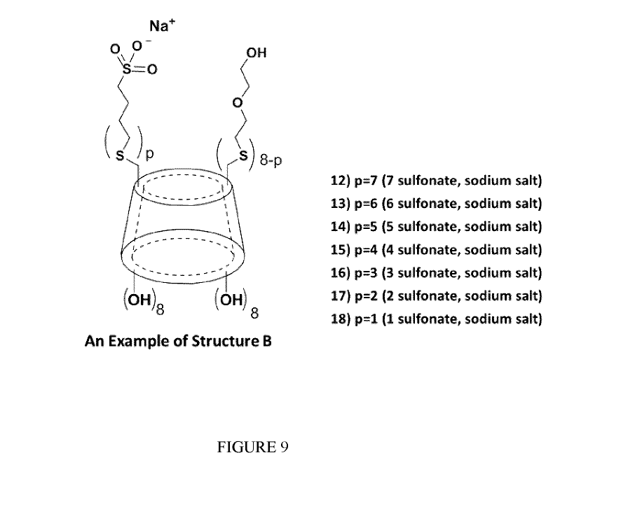

FIG. 9 shows an example of a selection of bifunctional compounds containing anionic groups (such as sulfonate) and non-targeting groups (such as polyethylene glycol, PEG). As with the targeting/anionic bifunctional libraries described above, the CDs in this example contain a complement of either anionic and/or non-ionic residues, for a total of 8 residues (for a γ-CD having 8 dextrin monomers). Thus, for a compound containing p=1 anionic residue, it would have a corresponding 8-p (i.e., 7) non-targeting (e.g., PEG) residues.

Compounds 12, 13, 14, 15, 16, 17, and 18 contain 7, 6, 5, 4, 3, 2 and 1 sulfonate residues, respectively.

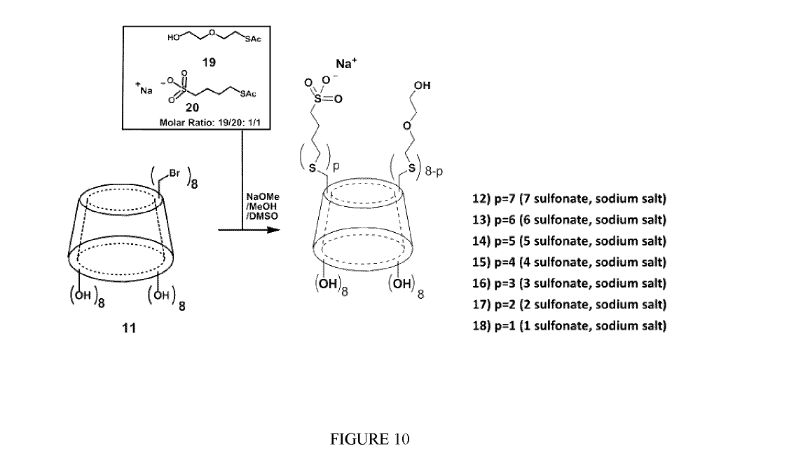

FIG. 10 shows an exemplary synthesis of a bifunctional library of γ-CD (m=8) containing sulfobutyl and non-targeting PEG groups.

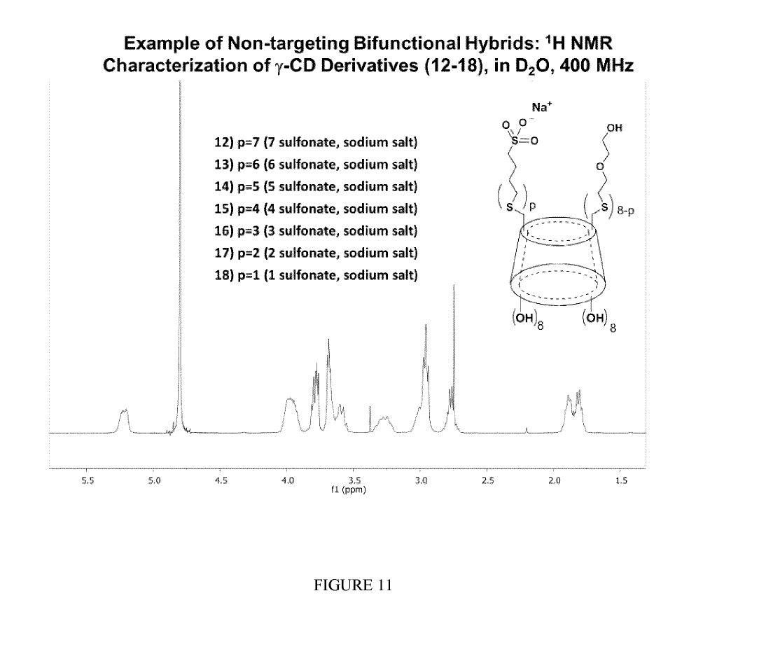

FIG. 11 shows 1H NMR spectrum of a bifunctional library of γ-CD (m=8) containing compounds 12-18

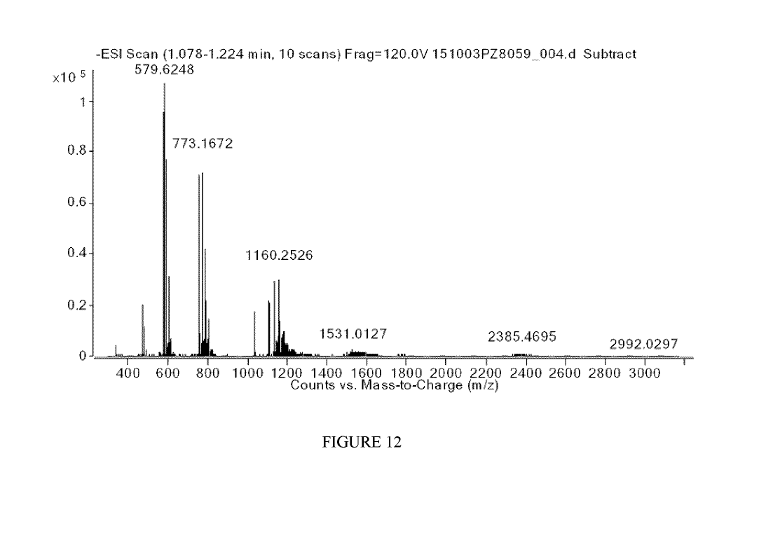

FIG. 12 shows ESI HRMS spectrum of a bifunctional library of γ-CD (m=8) containing compounds 12-18.

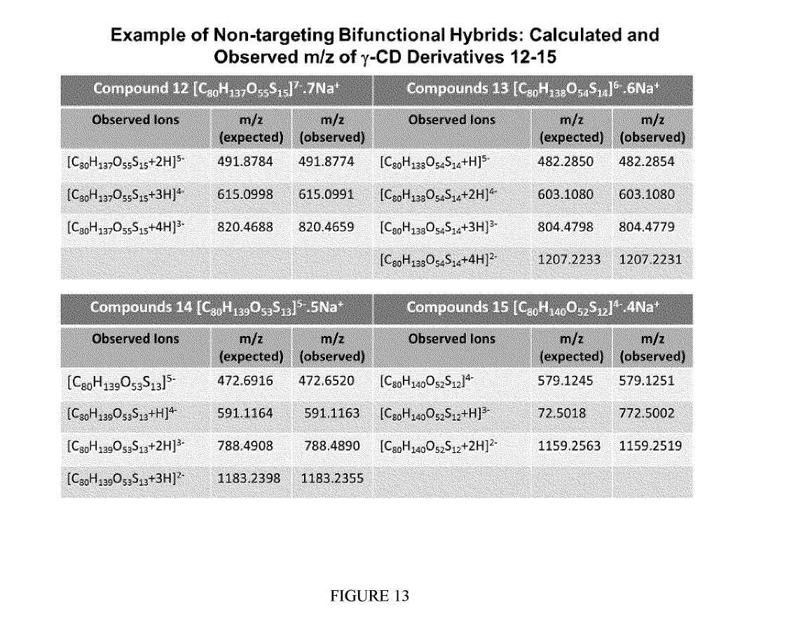

FIG. 13 shows ESI HRMS characterization of a bifunctional library of γ-CD (m=8) containing compounds 12-15.

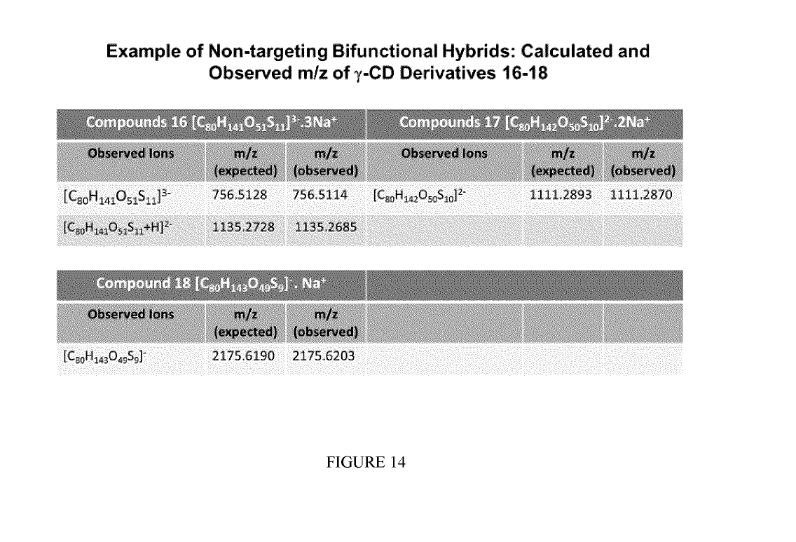

FIG. 14 shows ESI HRMS characterization of a bifunctional library of γ-CD (m=8) containing compounds 16-18.

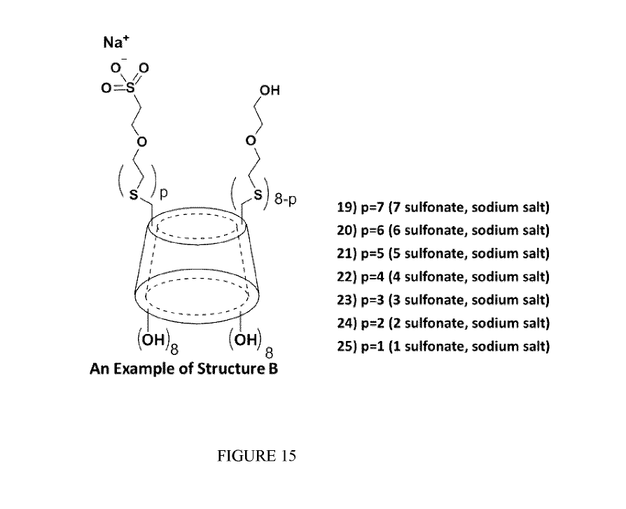

FIG. 15 shows another example of non-targeting bifunctional library containing compounds 19-25 that bear both the anionic sulfodiethylene glycol (SulfoDiPEG) and non-targeting diethylene glycol groups.

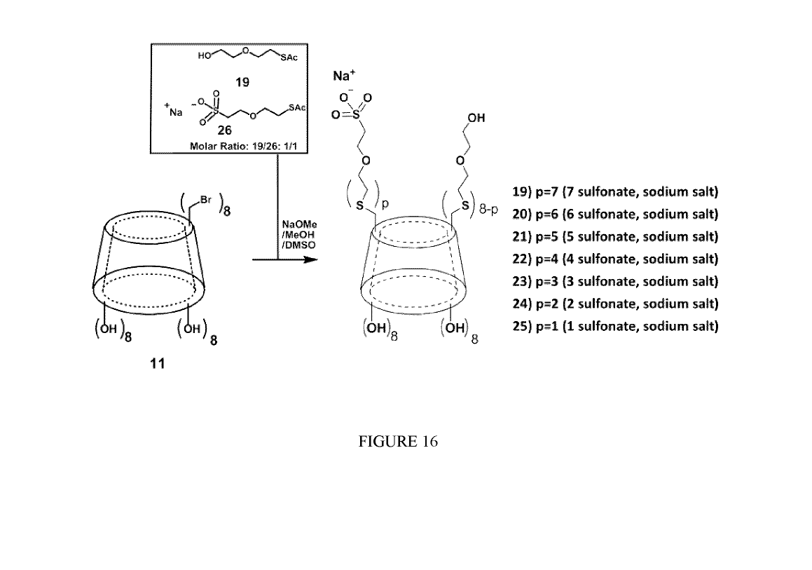

FIG. 16 shows an exemplary synthesis of the bifunctional library of γ-CD (m=8) containing compounds 19-25.

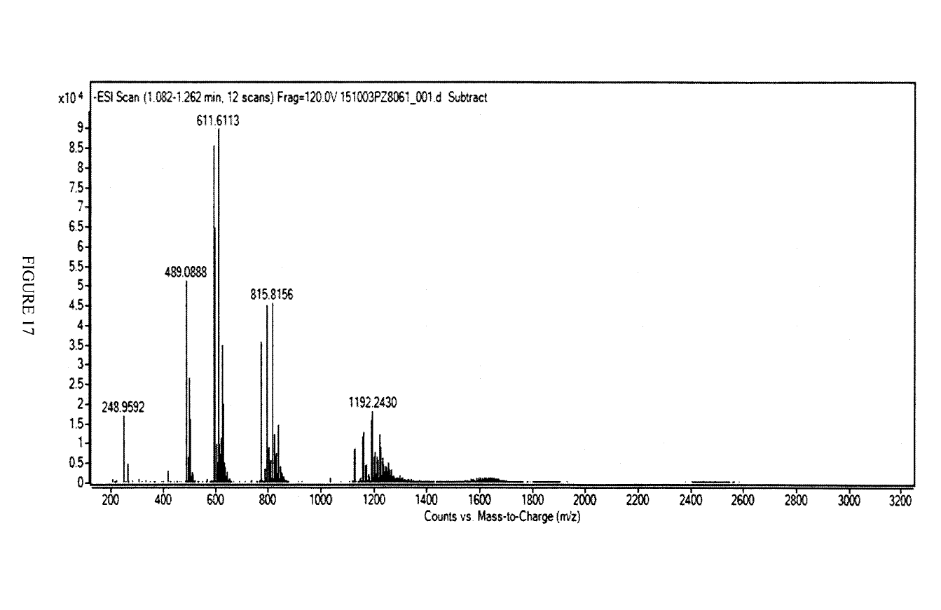

FIG. 17 shows ESI HRMS spectrum of the bifunctional library of γ-CD (m=8) containing compounds 19-25.

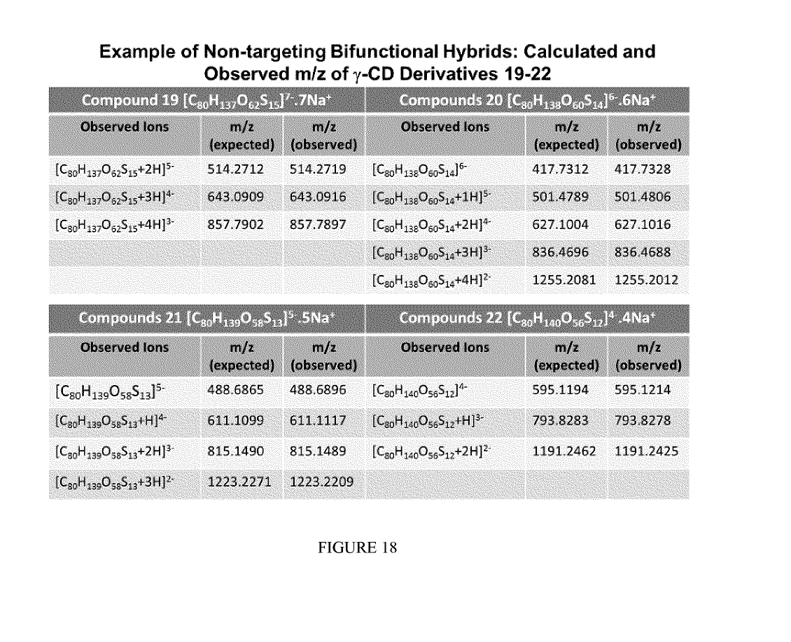

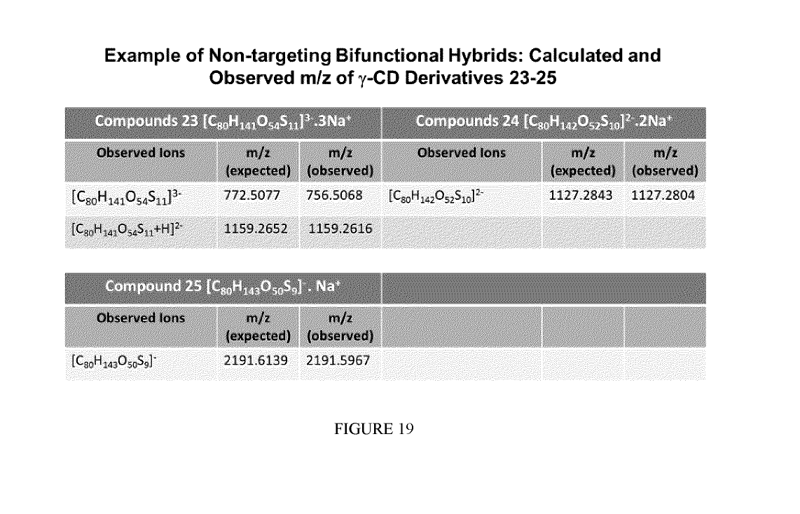

FIGS. 18 and 19 show ESI HRMS characterization of the bifunctional library of γ-CD (m=8) containing compounds 19-25.

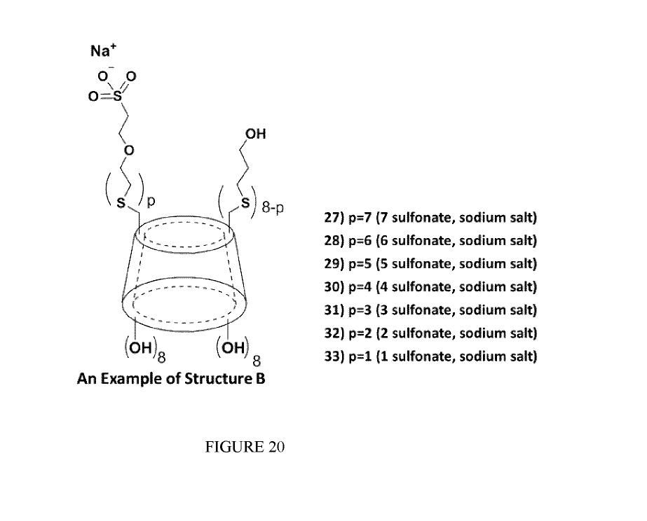

FIG. 20 shows third example of a selection of non-targeting bifunctional library containing compounds 27-33 that bear both the anionic sulfodiethylene glycol (SulfoDiPEG) and non-targeting hydroxypropyl groups.

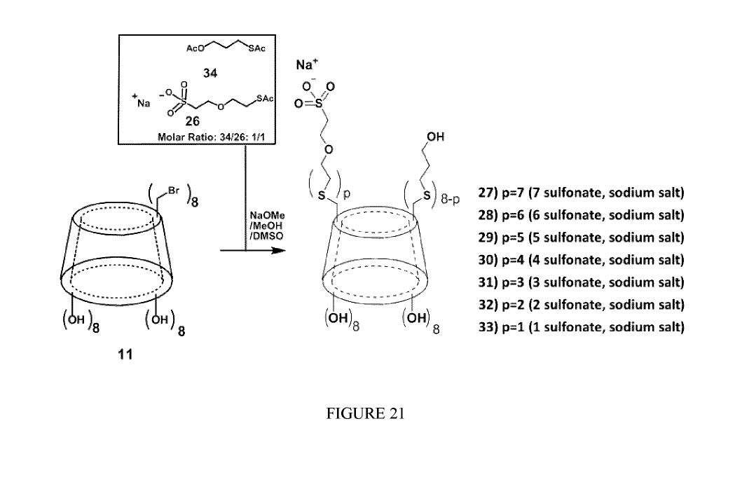

FIG. 21 shows an exemplary synthesis of the bifunctional library of γ-CD (m=8) containing compounds 27-33.

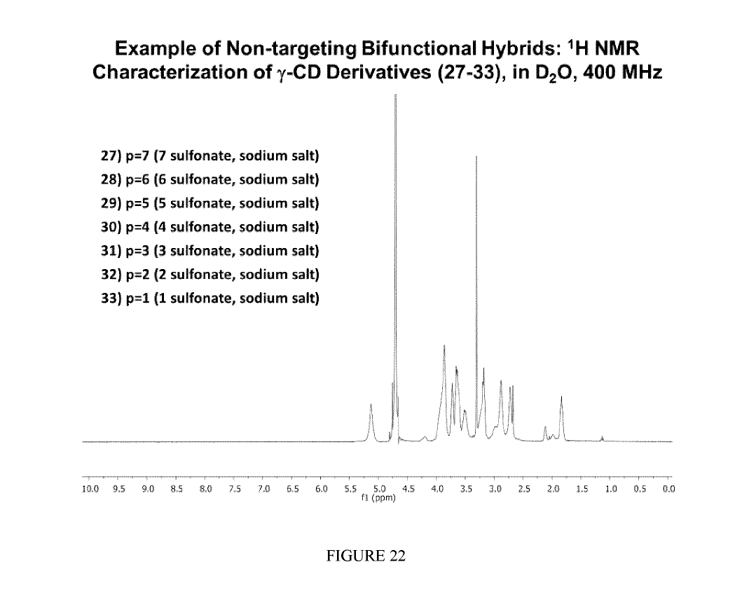

FIG. 22 shows ¹H NMR spectrum of a bifunctional library of γ-CD (m=8) containing compounds 27-33.

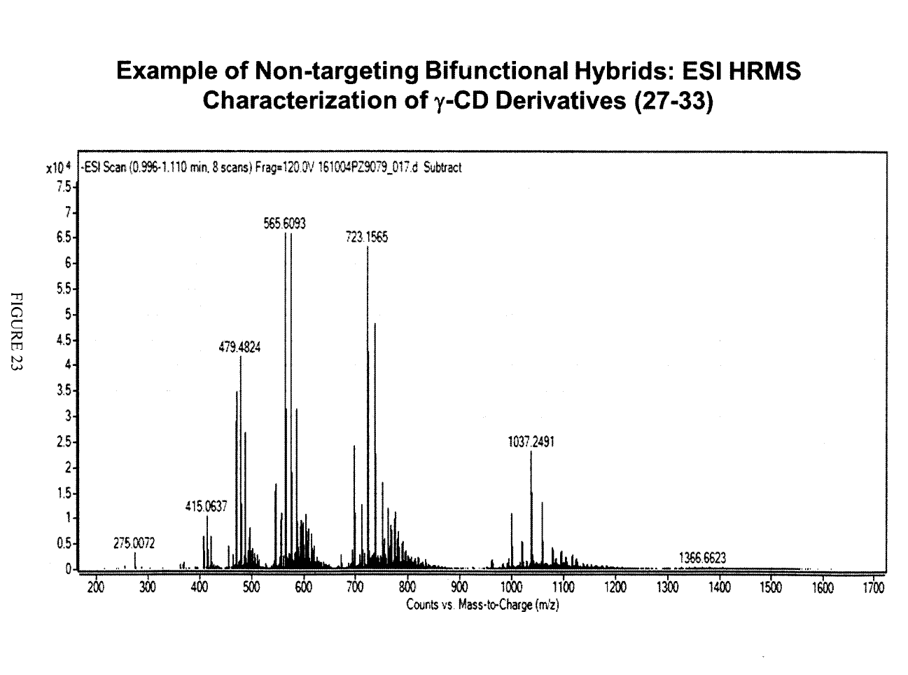

FIG. 23 shows ESI HRMS spectrum of the bifunctional library of γ-CD (m=8) containing compounds 27-33.

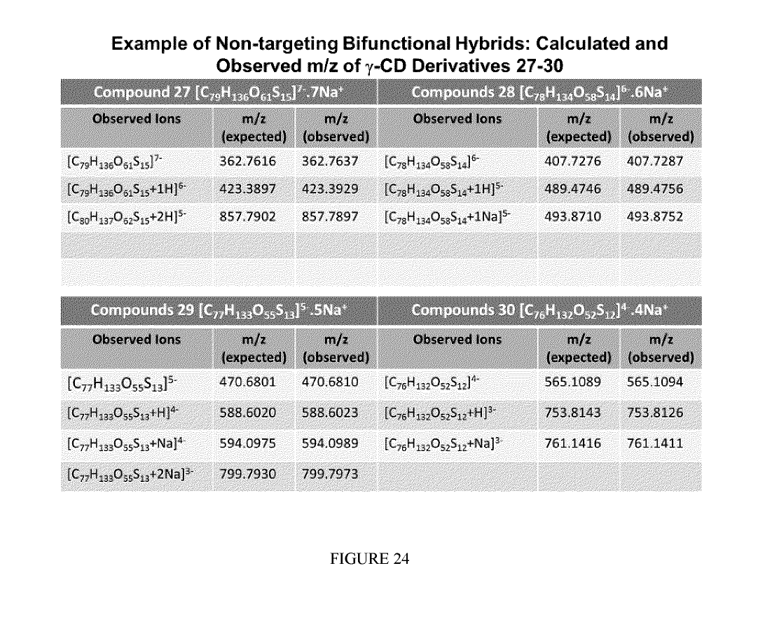

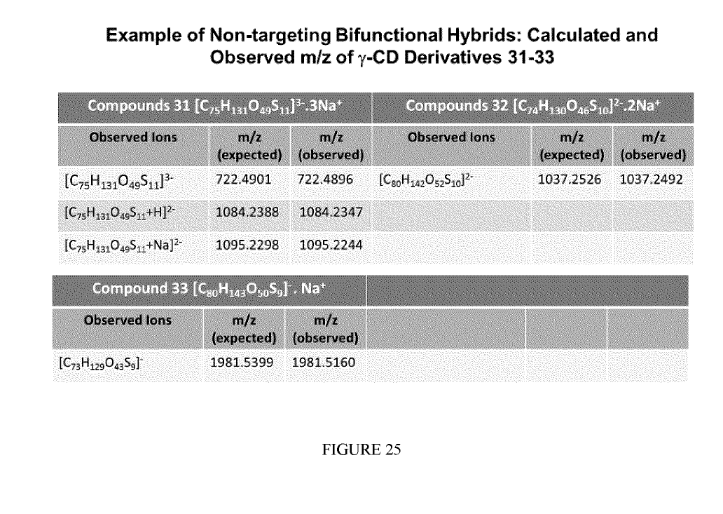

FIGS. 24 and 25 show ESI HRMS characterization of the bifunctional library of γ-CD (m=8) containing compounds 27-33.

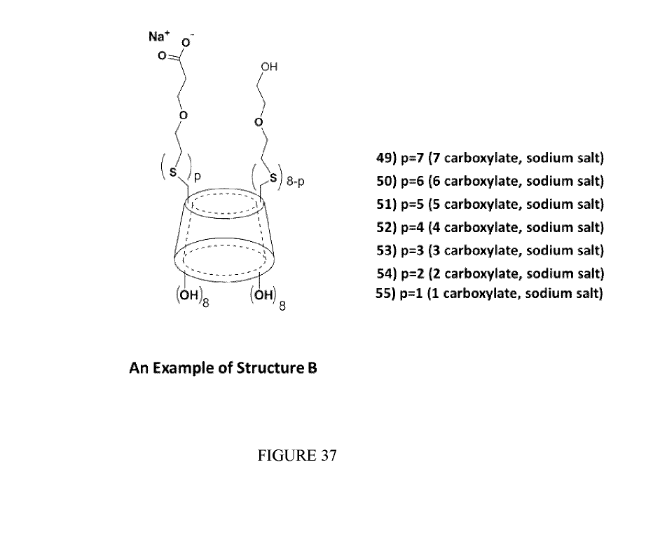

FIG. 37 shows a fourth example of a selection of non-targeting bifunctional library containing compounds 49-55 that bear both the anionic carboxydiethylene glycol (CarboxyDiPEG) and non-targeting diethylene glycol residues.

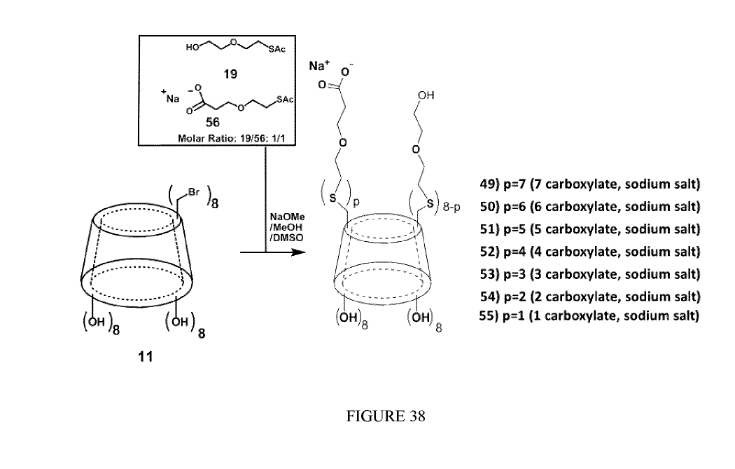

FIG. 38 shows an exemplary synthesis of the bifunctional library of γ-CD (m=8) containing compounds 49-55.

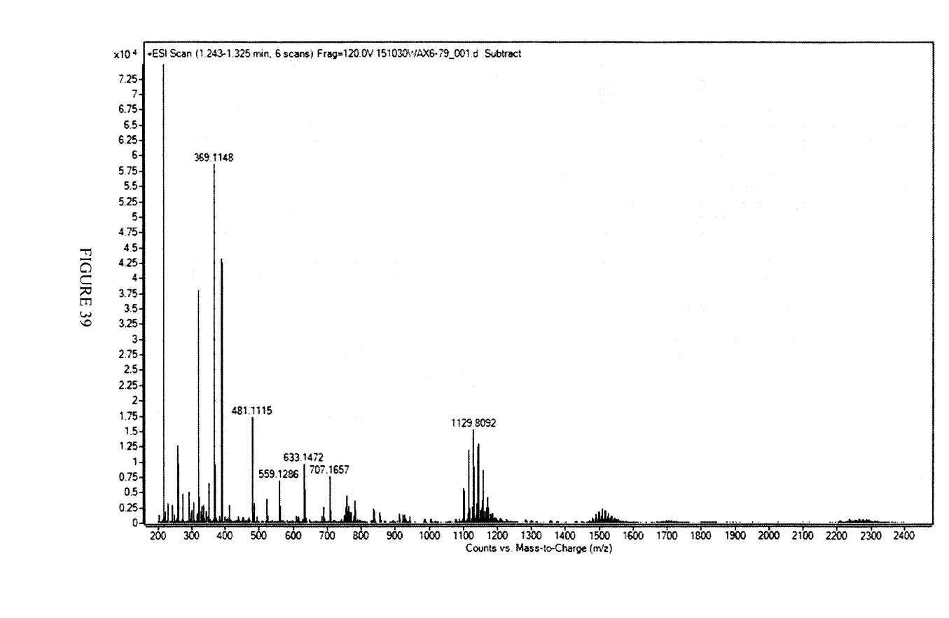

FIG. 39 shows ESI HRMS spectrum of the bifunctional library of γ-CD (m=8) containing compounds 49-55.

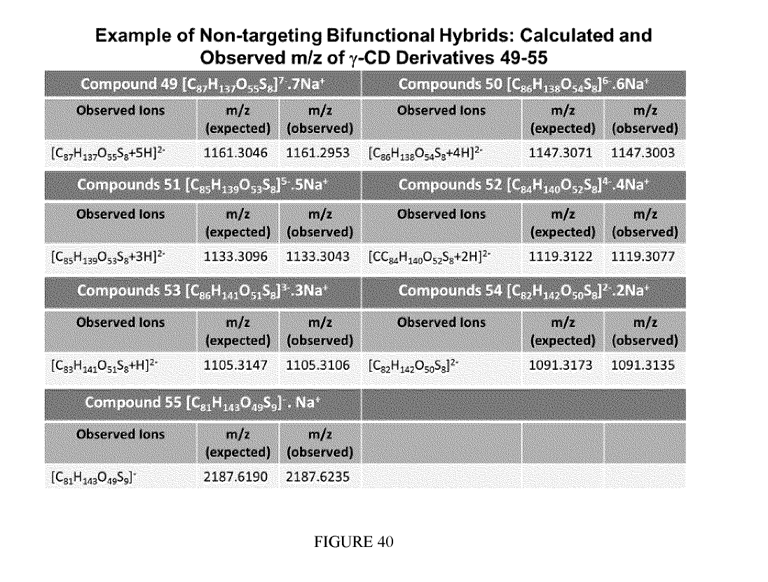

FIG. 40 show ESI HRMS characterization of the bifunctional library of γ-CD (m=8) containing compounds 49-55.

### Example 3

### Bifunctional Cyclodextrin Libraries Containing Anionic Functional Groups Via Different Linkers

In this example, the CD compounds contain anionic non-targeting residues but they are linked to CD core via different linkers.

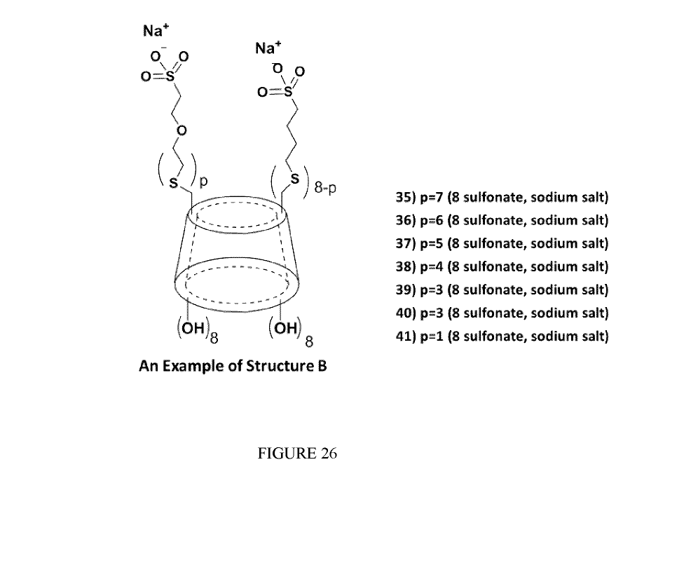

FIG. 26 shows an example of a selection of non-targeting bifunctional library containing compounds 35-41 that bear both the anionic sulfodiethylene glycol (SulfoDiPEG) and the anionic sulfobutyl groups. Two different linkers (PEG and butyl) are introduced to the same cyclodextrin scaffold using the one-pot synthesis methodology. The CDs in this example contain a total of either anionic residues, but the linkers consist of a mixture of tetramethylene (butyl) and diethylene glycol groups. Thus, for a compound containing p=1 butyl group, it would have a corresponding 8-p (i.e., 7) diethylene glycol group. The two types of linkers have difference hydrophobicity, which can affect their interaction with water as well as with the included guest molecules.

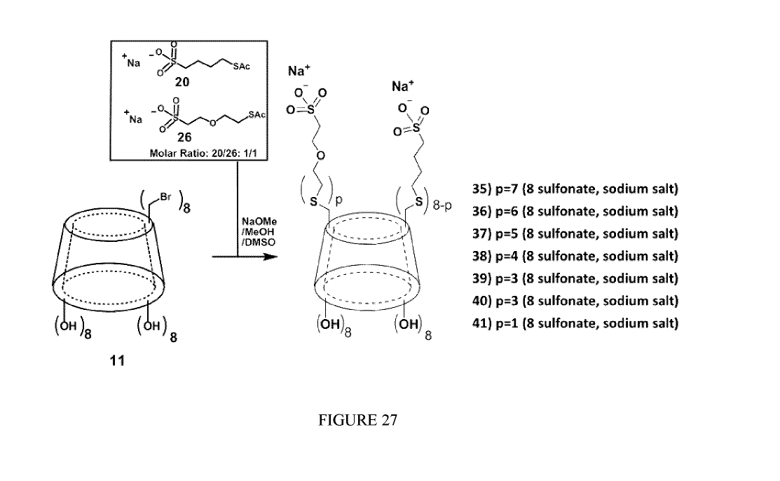

FIG. 27 shows an exemplary synthesis of the bifunctional library of γ-CD (m=8) containing compounds 35-41.

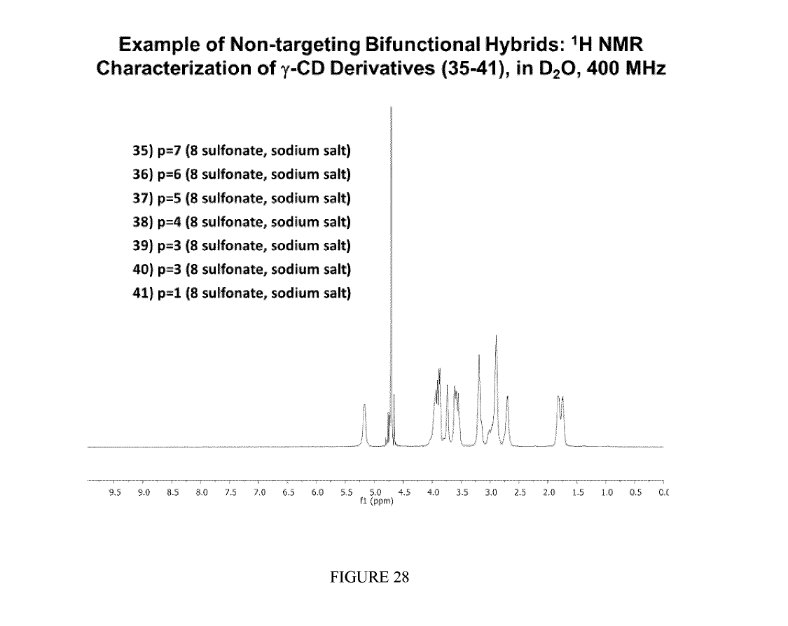

FIG. 28 shows ¹H NMR spectrum of a bifunctional library of γ-CD (m=8) containing compounds 35-41.

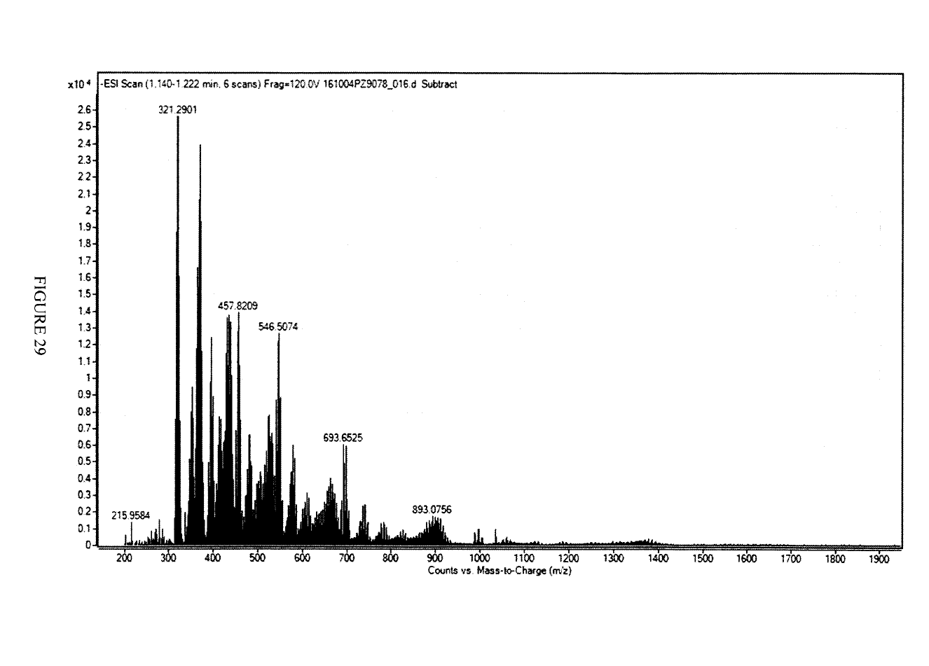

FIG. 29 shows ESI HRMS spectrum of the bifunctional library of γ-CD (m=8) containing compounds 35-41.

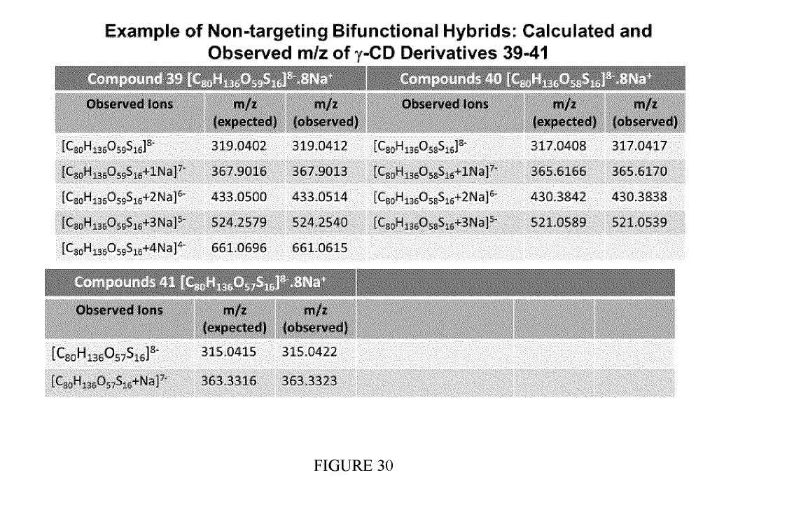

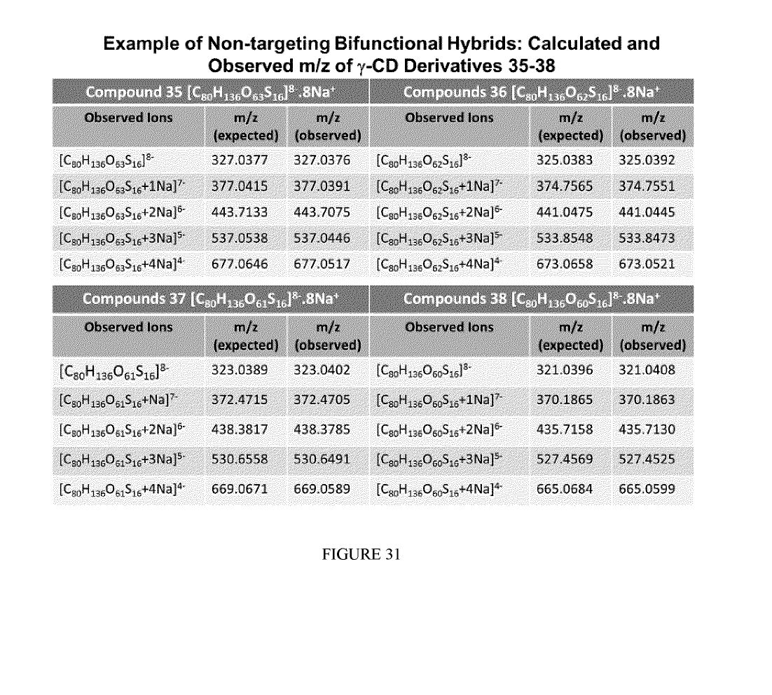

FIGS. 30 and 31 show ESI HRMS characterization of the bifunctional library of γ-CD (m=8) containing compounds 35-41.

### Example 4

### Inclusion Studies of Targeting and Non-Targeting Bifunctional Libraries With Commercial Medicines

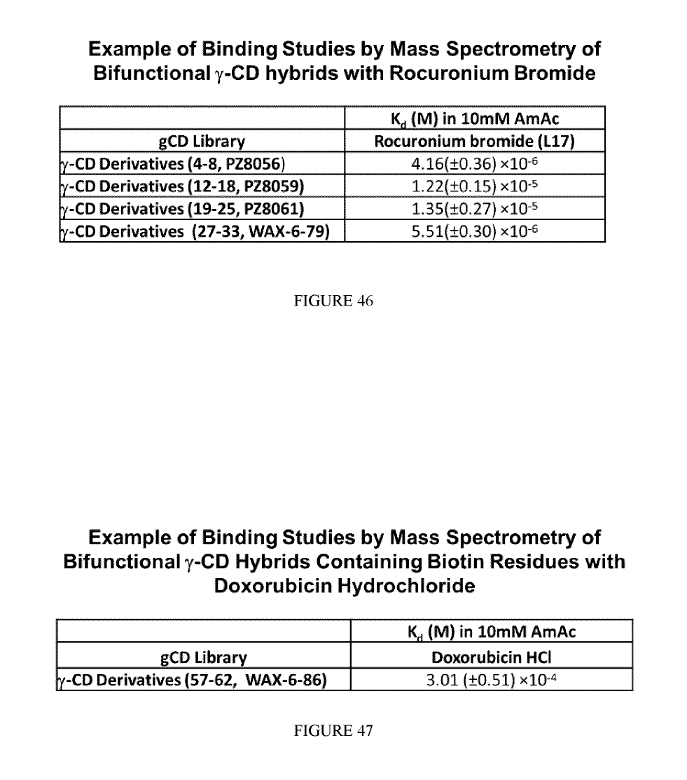

FIG. 46 shows the results of inclusion studies with commercial medicines by ESI mass spectrometry. As can be seen, the synthesized bifunctional cyclodextrin libraries can effectively bind to commercial medicines with different affinities. The commercial medicine employed in these examples is rocuronium bromide, but it can be any organic/inorganic compounds. Both targeting and non-targeting CD compounds have shown to bind to commercial medicines. The selected examples of libraries for binding studies include those containing compounds 4-8, 12-18, 19-25 and 27-33, respectively. The measured dissociation constant (K_{d}) with rocuronium bromide by ESI mass spectrometry vary depending on the functional groups present in the CD.

FIG. 47 shows another example of binding with doxorubicin hydrochloride. The selected library is the one with targeting capability containing biotin (compound 57-62). The measured dissociation constant (K_{d}) by ESI mass spectrometry with doxorubicin hydrochloride falls in the applicable range in drug formulation.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A multifunctional cyclodextrin dendrimer of the structure: 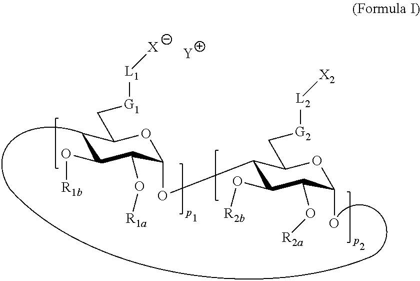
wherein
X⁽⁻⁾ is one or more negatively charged moieties,
Y⁽⁺⁾ is one or more counter cations,
X₂ is one or more neutral moieties,
L₁ and L₂ are each one or more linkers,
G₁ and G₂ are each a bond or are one or more bridging groups,
R1a, R1b, R2a and R2b are one or more substituents and can be the same or different, and each of p1 and p2 is at least 1, and p1+p2=6, 7, or 8;
where if p1>1, each of G₁, L₁, X⁽⁻⁾, Y⁽⁺⁾, can be the same or different from other G₁, L₁, X⁽⁻⁾, Y⁽⁺⁾, respectively; and
where if p2>1, each G2, L2, X2, can be the same or different from other G2, b, X2, respectively.

2. The multifunctional cyclodextrin dendrimer of claim 1, wherein Y⁽⁺⁾ is Na⁺.

3. The multifunctional cyclodextrin dendrimer of claim 1, wherein X⁽⁻⁾ is —CO₂— or —SO₃—, or —PO₃²⁻

4. The multifunctional cyclodextrin dendrimer of claim 1, wherein G₁ and/or G₂ is —S—.

5. The multifunctional cyclodextrin dendrimer of claim 1, wherein L₁ and/or L₂ is/are:
a) —(CH₂)k-, where k is 1 to 11, optionally 1 to 6;
b) 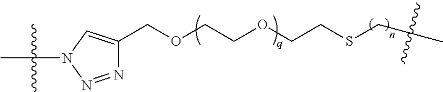
where q is 0 to 20 and n is 1-5, optionally 1-11; or
c) 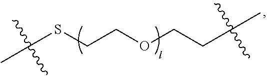
where 1 is 0-20.

6. The multifunctional cyclodextrin dendrimer of claim 1, wherein R₁ₐ, R_{1b}, R₂ₐ and R_{2b} are the same or different and are H, optionally substituted C1-C18 alkyl, or optionally substituted C1-C18 acyl.

7. The multifunctional cyclodextrin dendrimer of any one of claims 1 to 6, wherein X₂ is a targeting functional group.

8. The multifunctional cyclodextrin dendrimer of claim 7, wherein the targeting functional group is biotin, folic acid, lactose, N-acetyl-lactosamine, D-glucose, D-mannose, N-acetyl-D-glucosamine, L-fucose, N-acetyl-D-glucosamine, N-acetylneuraminic acid or the like.

9. The multifunctional cyclodextrin dendrimer of claim 1, wherein X₂ is —H, —OH, —NR₂, —CO₂NR₂, —CN or the like, where R is H, or a C1-C4 alkyl group.

10. The multifunctional cyclodextrin dendrimer of claim 1, which is: 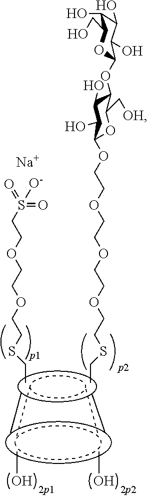
wherein p1+p2 is 6, 7 or 8.

11. The multifunctional cyclodextrin dendrimer of claim 10, wherein p1+p2 is 6, where p1 and p2 are 1 to 5.

12. The multifunctional cyclodextrin dendrimer of claim 10, wherein p1+p2 is 7, where p1 and p2 are 1 to 6.

13. The multifunctional cyclodextrin dendrimer of claim 10, wherein p1+p2 is 8, where p1 and p2 are 1 to 7.

14. The multifunctional cyclodextrin dendrimer of claim 1, which is one of A-F: 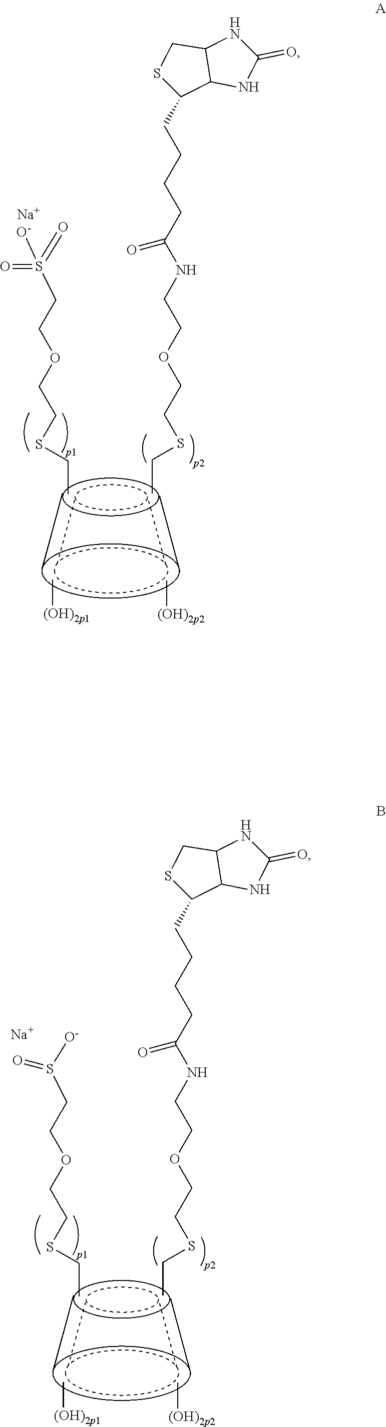 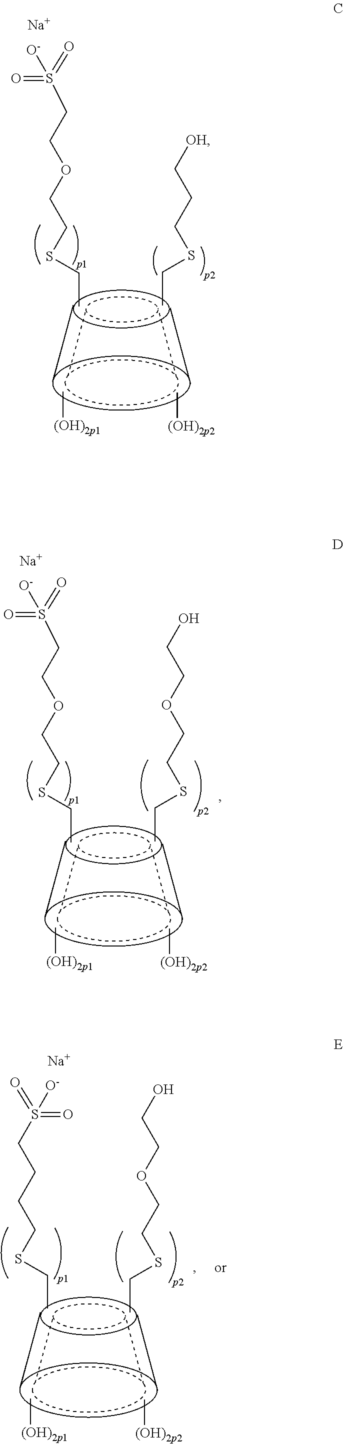 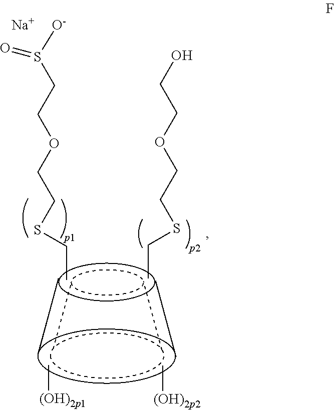
wherein p1+p2 is: a) 6, where p1 and p2 are 1 to 5; b) 7, where p1 and p2 are 1 to 6; or c) 8, where p1 and p2 are 1 to 7.

15.-20. (canceled)

15. A method of synthesizing a multifunctional cyclodextrin dendrimer of claim 1, comprising:
a) providing a per-6-substituted cyclodextrin with a leaving group at 6-position;
b) reacting the per-6-substituted cyclodextrin in a) with a first residue and a second residue;
c) obtaining the compound, wherein the first and/or second residues comprises one or more linker groups, one or more bridging groups and one or more functional groups.

16. The method of claim 15, wherein the one or more bridging groups is/are —S—.

17. The method of claim 15, wherein the one or more linker groups is/are
a) —(CH₂)k-, where k is 1 to 11, optionally 1 to 6;
b) 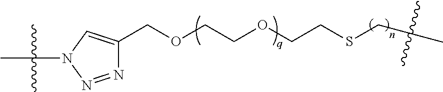
where q is 0 to 20 and n is 1-5, optionally 1-11; or
c) 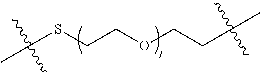
where 1 is 1-20.

18. The method of any one of claim 15, wherein the one or more functional groups on the first residue is/are —CO₂— or —SO₃— or —PO₃²⁻.

19. The method of claim 15, wherein the one or more functional groups on the second residue is/are biotin, folic acid, lactose, N-acetyl-lactosamine, D-glucose, D-mannose, N-acetyl-D-glucosamine, L-fucose, N-acetyl-D-glucosamine, N-acetylneuraminic acid or the like, or —OH, —NR₂, —CO₂NR₂, or the like, where R is H, or a C1-C4 alkyl group.

20. A method of drug delivery to a subject comprising administering to said subject a multifunctional cyclodextrin dendrimer of claim 1 together with the drug.

27.-28. (canceled)
